(19) United States
(12) Patent Application Publication
(10) Pub. No.: US 2006/0229709 A1
(43) Pub. Date: Oct. 12, 2006

(54) Vascular graft

(76) Inventors: Liam Gerald Morris, Galway (IE); Timothy M. McGloughlin, County Limerick (IE); Patrick Delassus, County Galway (IE); Michael Thomas Walsh, County Limerick (IE); Thomas Patrick O'Brien, County Cork (IE); John Anthony Callanan, County Galway (IE)

Correspondence Address: JACOBSON HOLMAN PLLC, 400 SEVENTH STREET N.W., SUITE 600, WASHINGTON, DC, 20004, US

(21) Appl. No.: 11/391,474

(22) Filed: Mar. 29, 2006

Related U.S. Application Data
(60) Provisional application No. 60/666,193, filed on Mar. 30, 2005.

(51) Int. Cl. A61F 2/06

(52) U.S. Cl. 623/1.31; 623/1.35

(57) ABSTRACT

A vascular graft comprising a proximal section, iliac distal legs and a bifurcation blending section (7) between the proximal section and the distal legs. The cross-sectional area of the proximal section at the bifurcation point is less than or equal to the sum of the two cross sectional areas of both iliac legs. The blending section (7) generates a smooth transition from the proximal section to both iliac legs which minimizes wave reflections by ensuring that the area ratio at the bifurcated junction (7) is as close to unity or greater than unity as possible. The blending section (7) defines a first lumen for fluid flow from the proximal section into the first distal leg, and a separate second lumen for fluid flow from the proximal section into the second distal leg. The two lumen are separated by means of a gradual flow which separates the fluid flow from the proximal section into each lumen. The distal legs are connected to the blending section (7) at the bifurcation region to form a substantially “Y”-shaped graft.

### INTRODUCTION

This invention relates to vascular and endovascular grafts, such as for abdominal aortic aneurysms (AAA) or any other vascular disease, such as stenois or blocked arteries, or for the airways of the lung.

An aneurysm is an abnormal localised sac or an irreversible dilation caused by a weakness (decreased elastin) of the arterial wall. The arterial wall comprises three layers: the intima (inner wall), the media (middle wall) and the adventitia (outer wall). Damage to the media gives rise to AAA. Aneurysms are classified as either fusiform or saccular. In the fusiform case the entire circumference is affected, while one side is affected in the saccular form. Aneurysms can result from accidents, arteriosclerosis, high blood pressure, or a congenital disease. Over time the vessel wall loses its elasticity and the normal blood pressure in the aneurysm sac can lead to rupture of the vessel wall, which causes internal bleeding and eventual death in many cases. Even if the vessel wall does not rupture, a large aneurysm can impede circulation and promote unwanted blood-clot formation.

Most patients do not indicate any specific symptoms that they have an abdominal aortic aneurysm. The problem is normally diagnosed during routine medical examination or when diagnostic imaging such as x-ray is performed for other reasons.

There are currently two surgical treatments for acute AAA: open surgery or minimally invasive repair also known as the endovascular repair procedure. The objective of both methods is to isolate the aneurysm sac from systemic blood pressure and flow so as to minimize the risk of arterial wall rupture. Clinical success is defined by the “total exclusion” of the aneurysm. As a result most AAAs should stabilize or shrink. Traditional surgical repair involves opening the chest or abdomen, gaining temporary vascular control of the aorta, and below the lesion, opening the aneurysmal sac and suturing a prosthetic synthetic graft to the healthy aorta within the aneurysm itself. The outcome of standard surgical Abdominal Aortic Aneurysm (AAA) repair has proven to be excellent, with mortality rates in the range of 3% to 5%. However, standard AAA repair is not perfect, and the quality of life after this repair is impaired by postoperative pain, sexual dysfunction, and a lengthy hospital stay resulting in high health costs. These negative effects are related to the large incision and extensive tissue dissection. Mortality and morbidity increase with the presence of associated diseases and a mortality rate of 60% has been reported for high-risk patients. The standard repair is also extremely difficult in patients with a prior history of abdominal operations where extensive scarring and infection may be present. Endovascular grafting is an alternative treatment to standard open aneurysm repair. This treatment involves a surgical exposure of the common femoral arteries where the endovascular graft can be inserted by an over-the-wire technique. This is where the endovascular graft is positioned onto a catheter (tubing based delivery system) over a guidewire. Using x-ray imaging this tubing based delivery system containing the endovascular graft is introduced via the femoral artery and positioned inside the aneurysm as shown in Fig. A.

The graft itself is a synthetic material often supported with a metal (typically nitinol or 316L stainless steel) endoskeleton. Graft fixation is often achieved by the stent which creates a fixation at the proximal end by barbs or by a stent portion that is uncovered by graft material. Distal end fixation is attained by friction within the branch or iliac arteries. Such endovascular treatments offer the economic advantages of short hospital stays or even treatment as an outpatient, as well as elimination of the need for postoperative intensive care and are, therefore, extremely attractive to both patients and physicians.

Fig. B shows a typical 3D line drawing of a prior art bifurcated stent graft device comprising a stent mesh integrated into the graft. Referring to Fig. C, the Ancure® stent graft is a bifurcated, non supported stent-graft with proximal and distal “hook like” fixation devices made of Elgiloy™. The Zenith™ stent graft consists of a main body and is comprised of an aortic section, one short iliac limb (contralateral limb) and one long iliac limb (ipisalateral limb), as shown in Fig. D. The main graft component consists of woven polyester and is fully stented with self-expanding stainless steel z-stents. It also contains an uncovered suprarenal stent with hooks, which aids in fixation. The AneuRx® AAA stent graft system is a modular design with self-expanding stents with a thin wall polyester graft material, as shown in Fig. E.

Referring to Fig. F, U.S. Pat. No. 6,685,738 describes a bifurcated stent graft device comprising a proximal end, which bifurcates into a first frustoconical leg transition with a dependant iliac leg. There is also a second frustoconical leg transition, which joins up to a dependant iliac leg. For modular design stent grafts the second iliac leg is connected separately via the frustoconical leg transition, which may have barbs to help firmly connect second leg to leg transition. The proximal stent is typically implanted within the vasculature below the renal arteries in the aorta such that the main body and leg transitions are positioned within the aorta main portion and with dependant first and second leg each positioned within respective iliac arteries.

Other grafts are described in U.S. Pat. No. 6,695,875, U.S. Pat. No. 6,576,009, U.S. Pat. No. 6,224,609, U.S. Pat. No. 6,773,454 and WO99/40875. The first endovascular repair of abdominal aortic aneurysms was performed more than a decade ago. Preliminary results have been promising with short-term results comparable with conventional surgical repair. Long-term results are not so encouraging with stent graft migration, endoleaks, material failure and aneurysm rupture all being reported.

Secure proximal fixation of stents for AAA is pivotal to the long-term success of the endovascular procedure. Problems due to stent graft fixation can lead to endoleaks and stent graft migration, leaving the aneurysm exposed to systemic blood pressure. A well-known complication with this endovascular procedure is the late migration of the graft in which most of the devices are diagnosed after the first 12 months after the procedure. The effect of the migration is to expose the aneurysm sac to systemic blood pressure and flow, which if left untreated has serious consequences for the patient. Endoleaks lead to the total volume increase of the aneurysm due to the direct arterial flow into the aneurysm. This generates systemic pressurization of the aneurysm sac that eventually leads to expansion and rupture. There are five types of endoleaks: Type I—originating at the attachment sites in the aneurysm neck or iliac arteries; Type II—retrograde flow into the aneurysm sac through the lumbar arteries or inferior mesenteric artery (IMA); Type III—modular disassociation such as fabric tears or an inadequate seal for modular devices; Type IV—graft material porosity and Type V—Endotension.

Gradual enlargement of the proximal neck has been reported after stent graft repair with an enlargement rate of approximately 1 mm/year. Usually the proximal end of an endograft is oversized by 2 to 4 mm and the significance of this dilation is that the attachment mechanism loses its radial force and therefore starts to migrate.

Endovascular stent graft fatigue failures have been recognized in devices after aortic implantation. This fatigue failure leads to delayed hook fractures, metallic stent fractures, suture disruptions, fabric erosion (caused by abrasion of the polyester woven fabric with the underlying stent) and late failure of aortic neck attachments.

Stent graft failures are known to occur at the bifurcation points. Stent graft thrombosis and micro-embolism are two complications associated with endovascular repair of AAA. Stent graft occlusion in the iliac legs has also been shown. Several cases of fatal multi-organ failures have been linked to micro-embolism.

Fig. G shows the geometry of various AAA configurations and the suitability of vascular and endovascular surgery. Depending on the location and extent of the aneurysm, Types A, B and C are generally suitable to both the endovascular and surgical procedure while Types D and E can only be treated surgically.

Fig. H shows the typical internal dimensions of AAA as determined pre-operatively by the Eurostar Data Registry System. Generally, for a population base there can be quite a wide range of dimensional variation. Symmetric and unsymmetric iliac artery set ups were found with the bifurcation angle θ varying considerably from 5° to 90°.

This invention is directed towards providing an improved vascular graft.

### STATEMENTS OF INVENTION

According to the invention there is provided a vascular graft comprising:
a proximal section;
a first distal leg; and
a second distal leg;
the cross-sectional area of the proximal section being less than or equal to the sum of the cross-sectional area of the first distal leg and the cross-sectional area of the second distal leg.

In one embodiment of the invention the graft comprises a blending section between the proximal section and the distal legs. Preferably the blending section defines a first lumen for fluid flow from the proximal section to the first distal leg. Ideally the blending section defines a second lumen for fluid flow from the proximal section to the second distal leg. Most preferably the first lumen is separate from the second lumen. The longitudinal axis of the first lumen may be substantially parallel to the longitudinal axis of the second lumen.

In one case the first distal leg and the second distal leg are connected to the blending section at a bifurcation region. Preferably the graft is substantially “Y”-shaped.

At least one of the distal legs may be formed integrally with the blending section. The blending section may be formed integrally with the proximal section. The graft may be of integral construction.

In one case the blending section comprises a gradual flow separator to separate flow from the proximal section into the first lumen and into the second lumen. An apex section may be incorporated in the blending section. The gradual flow separator may take the form of a parabolic, hyperbolic, elliptical, circular, Bezier or B-Spline shape or a combination of these curves. The apex section may take the form of a parabolic, hyperbolic, elliptical, circular, Bezier or B-Spline shape or a combination of these curves. The apex section and gradual flow separator may be connected as one and take the form of a parabolic, hyperbolic, elliptical, circular, Bezier or B-Spline shape or a combination of these curves.

In one embodiment the blending section provides for a small difference in cross-sectional area between the proximal section and the sum of the cross-sectional areas of the distal legs. The blending section may be between the proximal section and an apex of the graft. The blending section may be incorporated in either one or both of the distal legs.

In another case the cross-section of the proximal section, and/or of the gradual flow separator, and/or of the apex section, and/or of the distal leg is circular, elliptical, parabolic, hyperbolic, Bezier, B-spline shape or a combination of these curves.

In one embodiment of the invention the blending section is shaped to minimise pressure wave reflection back to the proximal section. The blending section may be shaped to minimise flow recirculation. The blending section may be shaped to minimise skewing of flow and secondary flow profiles throughout the graft.

In one case at least part of the graft tapers distally inwardly. At least part of the proximal section may taper distally inwardly. At least part of the distal leg may taper distally inwardly.

In another case at least part of the graft tapers distally outwardly. At least part of the distal leg may taper distally outwardly. The proximal section may be tapered. The distal legs may be bell-shaped.

In one case the first distal leg and the second distal leg are substantially symmetrical. In another case the first distal leg and the second distal leg are substantially asymmetrical. Eccentricity may be included.

In another embodiment the angle subtended between:
the longitudinal axis of the blending section; and
an axis extending through the centroid of the proximal end of the proximal section and through the centroid of the proximal end of the distal leg;
   is in the range of from 0° to 15°.

The graft may be of a material having elasticity properties matching those of a host vessel. The elasticity properties of the graft may vary from 0.1 MPa to 500 MPa. The elasticity characteristics may have viscoelastic or non-linear stress/strain properties.

In one case the graft is of a mono or multi-filament yarn material. The graft material may be a combination of polyester knit and polyurethane or silicone or any other biocompatible rubber or polymer material. The graft may be at least partially of a stretchable material.

In another case the stent material is a shape memory alloy, such as Nitinol, stainless steel or any other biocompatible metal or polymer. The graft may have a stented structure. The graft may have a partially stented structure with stents at the proximal and distal legs.

In one case the graft comprises struts from the proximal section to the distal legs. The graft may comprise a tissue based structure.

In one embodiment the graft is configured for treatment of Abdominal Aortic Aneurysms or any other vascular disease, such as stenois or blocked arteries, or for treatment of blockages in the airways of the trachea entering the lung. The graft may be configured for implantation by vascular surgery. The graft may bes configured for implantation by endovascular surgery. The graft may be modular, having different sized sections for the proximal and both distal legs exist for general and patient-specific anatomy sizes.

In a further aspect of the invention there is provided vascular graft comprising:
a proximal section;
a first distal leg;
a second distal leg; and
a blending section between the proximal section and the distal legs;
the first distal leg and the second distal leg being connected to the blending section at a bifurcation region;
the blending section defining a first lumen for fluid flow from the proximal section to the first distal leg and a second lumen for fluid flow from the proximal section to the second distal leg, the first lumen being separate from the second lumen;
at least one of the distal legs being formed integrally with the blending section.

According to the invention, there is provided a vascular graft comprising a proximal section and at least two distal legs, wherein the graft further comprises a blending section between the proximal section and the distal legs, the blending section being shaped to minimize one or more of:
pressure wave reflections back to the proximal section,
flow recirculation, and
skewing of flow and secondary flow profiles throughout the graft.

In one embodiment, the graft configuration is suitable for the treatment of Abdominal Aortic Aneurysms or any other vascular disease such as stenois or blocked arteries or for treatment of blockages in the airways of the trachea entering the lung.

In another embodiment, the graft is suitable for vascular surgery.

In a further embodiment, the graft is suitable for endovascular surgery.

In one embodiment, the total cross-sectional area of the distal legs is equal to or greater than that of the proximal section thus resulting in an area ratio (ratio of proximal to distal leg areas) of less than or equal to 1.

In another embodiment, the blending section provides for a small difference in cross-sectional area between the proximal section and the total cross-sectional area of the distal legs.

In a further embodiment, a bifurcation begins at the proximal end.

In one embodiment, the graft comprises a gradual flow separator.

In another embodiment, an apex section is incorporated in the blending section.

In a further embodiment, the gradual flow separator takes the form of a parabolic, hyperbolic, elliptical, circular, Bezier or B-Spline shape or a combination of these curves.

In one embodiment, the apex section takes the form of a parabolic, hyperbolic, elliptical, circular, Bezier or B-Spline shape or a combination of these curves.

In another embodiment, the apex section and gradual flow separator are connected as one and take the form of a parabolic, hyperbolic, elliptical, circular, Bezier or B-Spline shape or a combination of these curves.

In a further embodiment, eccentricity is included.

In one embodiment, the blending section is between the proximal end and an apex of the graft.

In another embodiment, the blending section is incorporated in either one or both of the distal legs.

In a further embodiment, the cross-sections of the proximal section, gradual flow separator section, apex section and distal legs may be circular, elliptical, parabolic, hyperbolic, beizer, B-spline in shape or a combination of these curve details.

In one embodiment, the proximal section is tapered.

In another embodiment, the distal legs are bell-shaped.

In a further embodiment, the graft is of a material having elasticity properties matching those of the host vessel.

In one embodiment, the elasticity properties of the graft varies from 0.1 MPa to 500 MPa.

In another embodiment, the elasticity characteristics have viscoelastic or non-linear stress/strain properties.

In a further embodiment, the graft is of a mono or multi-filament yam material.

In one embodiment, the graft material is a combination of polyester knit and polyurethane or silicone or any other biocompatible rubber or polymer material.

In another embodiment, the stent material is a shape memory alloy, stainless steel or any other biocompatible metal or polymer.

In a further embodiment, the graft has a stented structure.

In one embodiment, the graft has a partially stented structure with stents at the proximal and distal legs.

In another embodiment, the graft comprises struts from the proximal to distal legs.

In a further embodiment, the graft comprises a tissue based structure.

In one embodiment, the graft is of integral construction.

In another embodiment, the graft is modular, having different sized sections for the proximal and both distal legs exist for general and patient-specific anatomy sizes.

### BRIEF DESCRIPTION OF THE DRAWINGS

The invention will be more clearly understood from the following description of some embodiments thereof, given by way of example only, with reference to the accompanying drawings, in which:

FIG. 1 is a diagram showing diagrammatically the geometry of a vascular graft according to the invention;

FIG. 2(a) is a diagram showing the top view of a vascular graft according to the invention;

FIG. 2(b) is a 3D line diagram showing, in perspective, sections of the vascular graft;

FIG. 2(c) is a 3-D line diagram showing a lateral view section of part of the vascular graft;

FIG. 3(a) is a line diagram showing a top view of a vascular graft according to the invention with non-blended distal legs;

FIG. 3(b) is a line diagram showing a top view of a blended vascular graft according to the invention with blended distal legs;

FIGS. 4(a), 4(b) and 4(c) are line diagrams showing the sections associated with the lateral view of a vascular graft according to the invention along the proximal end;

FIGS. 5(a) and 5(b) are line diagrams showing the curve details for a blended bifurcation lateral section;

FIG. 6 is a line diagram showing dimensions of a vascular graft according to the invention;

FIG. 7 is a line diagram of the cross-sections along the proximal end prior to the apex of a vascular graft according to the invention;

FIG. 8 is a 3-D perspective view of a vascular graft according to the invention;

FIGS. 9(a) and 9(b) are diagrams showing a vascular graft according to the invention with a tapered section at the proximal end and a bell shaped configuration at the distal ends;

FIG. 10(a) is a diagram showing the position of a vascular graft according to the invention inside an AAA for endovascular treatment;

FIG. 10(b) is a diagram showing the position of the vascular graft sutured onto the AAA for vascular treatment;

FIG. 11 is a plot showing a comparison of detected pressure in a prior graft and a vascular graft according to the invention;

FIG. 12(a) is a diagram showing vector velocity profiles across the centre of a prior art graft, while FIG. 12(b) shows that for a vascular graft according to the invention;

FIG. 13 is a diagram showing the contour and in-plane velocity profiles for both a prior art graft and the vascular graft according to the invention before and after the apex;

FIGS. 14(a) and 14(b) are diagrams showing wall shear stress for grafts of the prior art and of the invention respectively; and

FIGS. 15(a) and 15(b) are plots of pressure along the wall of a prior art graft and of the vascular graft according to the invention respectively.

### DETAILED DESCRIPTION

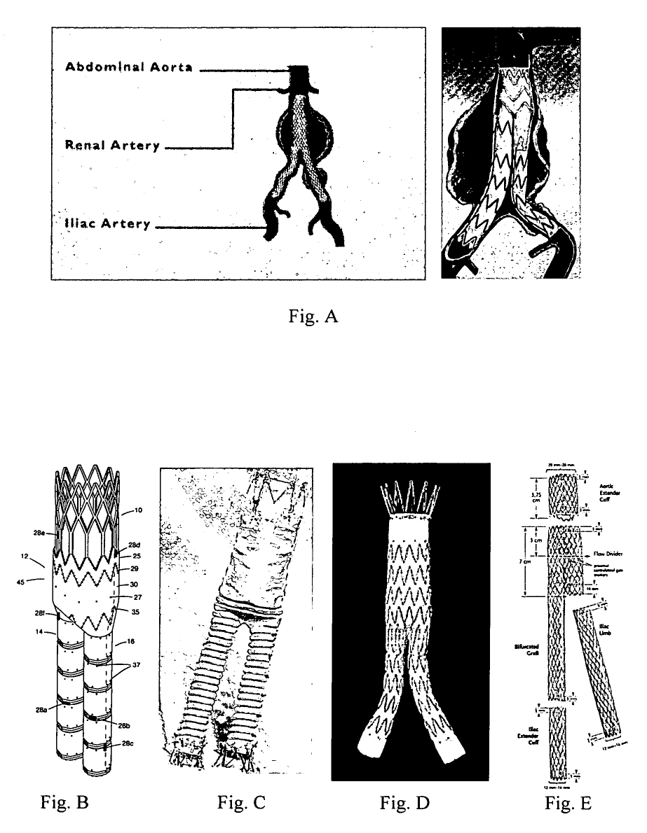

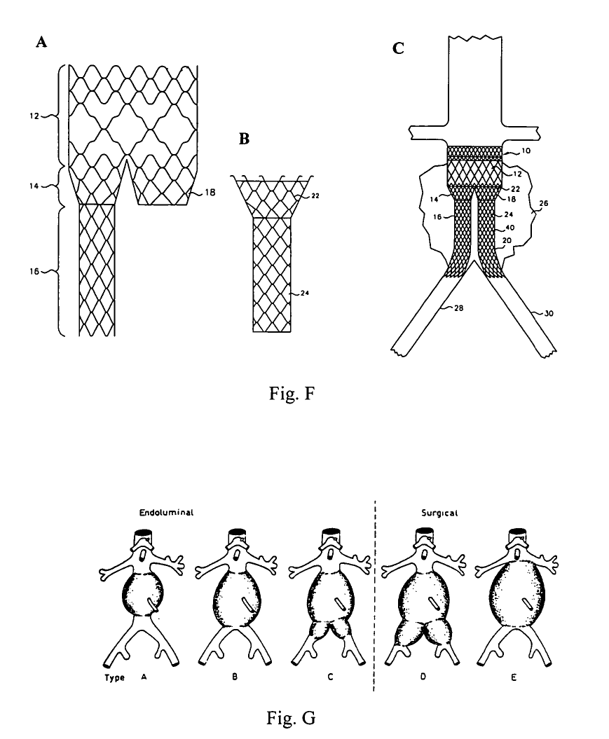

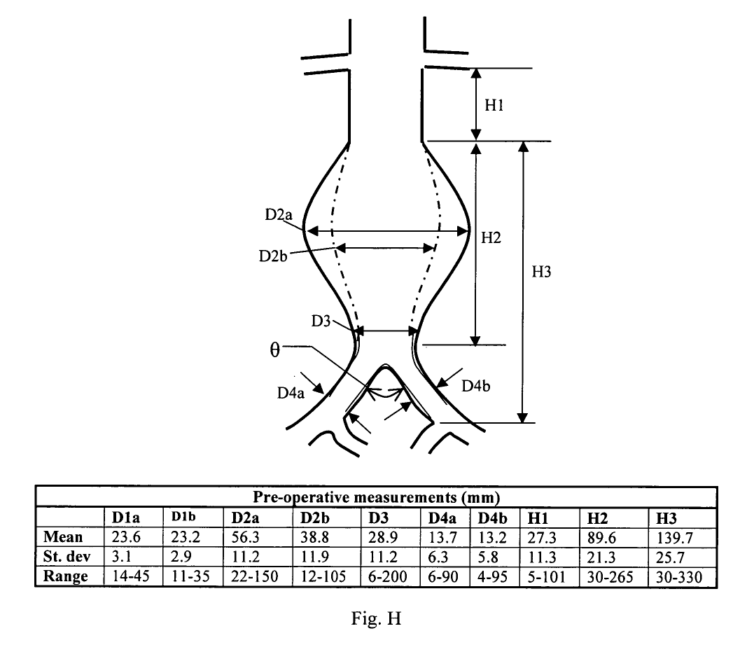

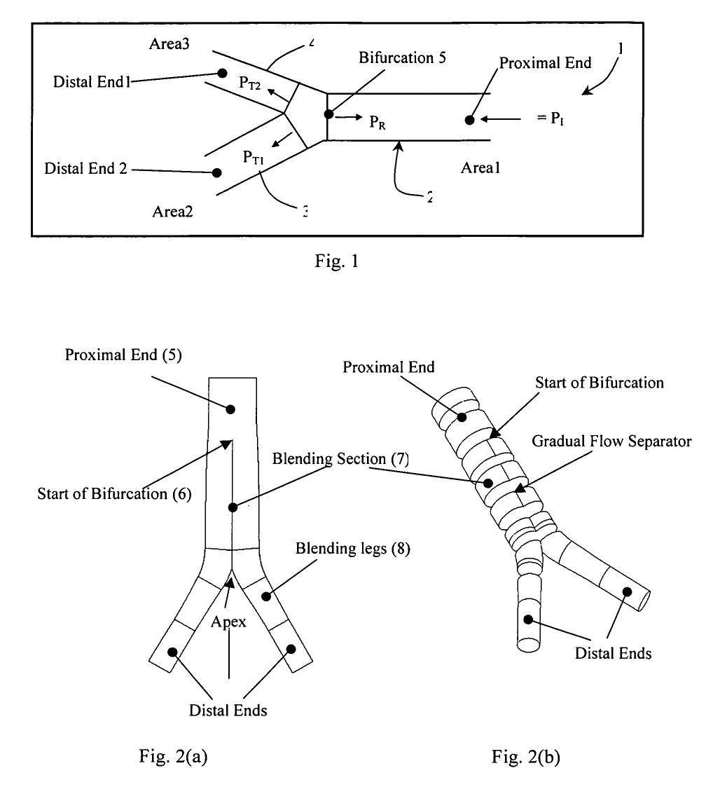

Referring to FIG. 1 a vascular graft 1 according to the invention is shown in diagrammatic form, and it comprises:
a proximal section 2;
iliac distal legs 3 and 4; and
a bifurcation blending section 5 between the proximal section 2 and the distal legs 3, 4.

The characteristics of the graft 1 are such that the cross-sectional area of the proximal section (Area 1) at the bifurcation point is less than or equal to the sum of the two cross sectional areas of both iliac legs 3, 4 (Area 2 and Area 3), i.e. Area1≦Area 2+Area 3. The area ratio of (Area2+Area 3)/Area1 should be as close to unity or greater as possible. This gives a total transmission of forward incident pressure wave (P_{I}) with no reflection at the junction. The blending section 5 minimizes wave reflections (P_{R}). This is very different from prior grafts which incorporate a shape at the bifurcation, which introduces a sudden cross sectional area change, at the bifurcation point from the proximal section to both iliac legs.

The blending section 5 generates a smooth transition from the proximal leg 2 to both iliac legs 3, 4 which minimizes wave reflections by ensuring that the area ratio at the bifurcated junction 5 is as close to unity or greater than unity as possible. This reduces the adverse effects subsequent to endovascular treatment of AAAs.

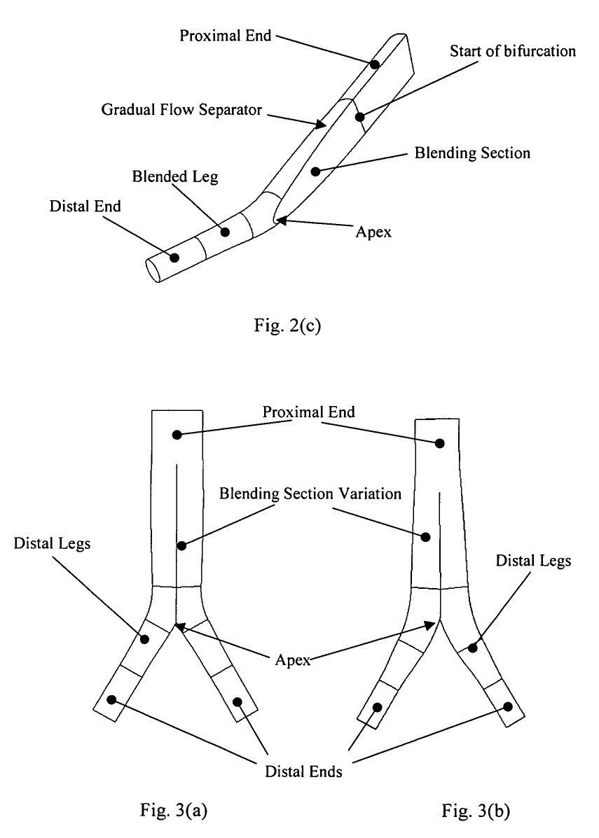

Based on the area ratio criteria, a blending section 7 was devised as shown in FIGS. 2(a), 2(b) and 2(c). This blending section 7 incorporates the following; a bifurcation which starts further upstream from the apex just below or at the proximal end 5. This creates a gradual flow separator, which aids in splitting the flow before the apex of the bifurcation, which is shown in FIGS. 2(b) and 2(c). This flow separator feature does not occur in prior art grafts.

The blending section 7 defines a first lumen for fluid flow from the proximal section 5 into the first distal leg, and a separate second lumen for fluid flow from the proximal section 5 into the second distal leg. The two lumen are separated by means of the gradual flow separator which separates the fluid flow from the proximal section 5 into each lumen. As illustrated in FIG. 2(a), the longitudinal axis of the two lumen are substantially parallel.

As illustrated in FIG. 2(b), the distal legs are connected to the blending section 7 at the bifurcation region to form a substantially “Y”-shaped graft. In this case the proximal section 5, the blending section 7 and the distal legs are all formed integrally.

Depending on the type of AAA, and the dimensions of AAA at the bifurcation and iliac legs as given in Fig. G and H respectively, non-blended or blended distal legs may be used as shown in FIGS. 3(a) and 3(b). Ideally a blended distal leg is preferred as this gives the smoothest transition for the blended section.

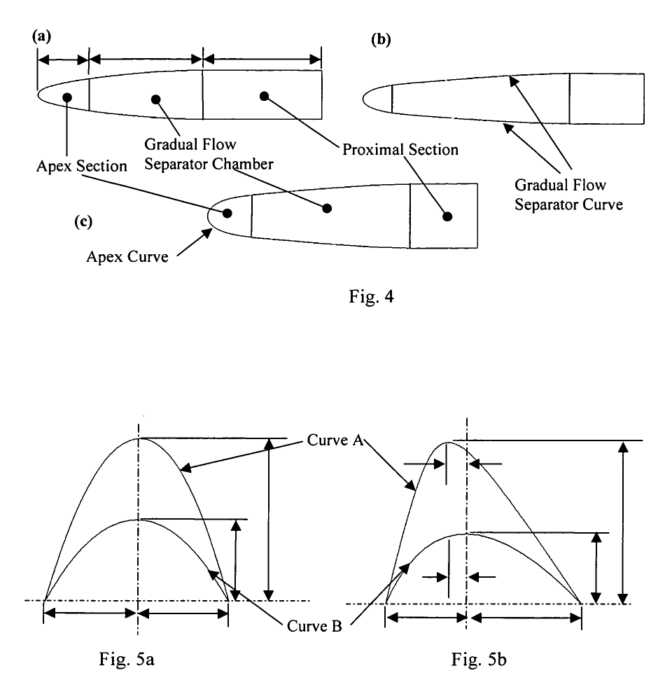

FIG. 4 shows a cross-section of the gradual flow separator along the proximal blending section. This consists of an apex section which blends into both distal legs, a gradual flow separator chamber just upstream from the apex section and the proximal section where a stent is positioned to attach against the wall of the vessel. FIGS. 4(a) to 4(c) shows how the apex curve and gradual flow separator curve can be varied from being a sharper apex curve (FIG. 4(a)) to a rounder apex curve (FIG. 4(c)). FIGS. 5(a) and 5(b) give the curve details and profiles for the apex and gradual flow separator curve. FIG. 5(a) shows a symmetrical gradual flow separator along the proximal blending section. Various curve details can be applied as shown by Curves A and B. These curves can take the form of a parabola, hyperbola, elliptical, circular, Bezier or B-Spline curves. These curves can be applied to the apex curve only where a parabolic, hyperbola, elliptical, Bezier or B-Spline curve is applied separately along the gradual flow curve. The curves given in FIG. 5 may be applied to both the apex and gradual flow separator curve together as one. Eccentricity can be applied to the curves as given by FIG. 5(b).

The distal legs may be symmetrical or asymmetrical.

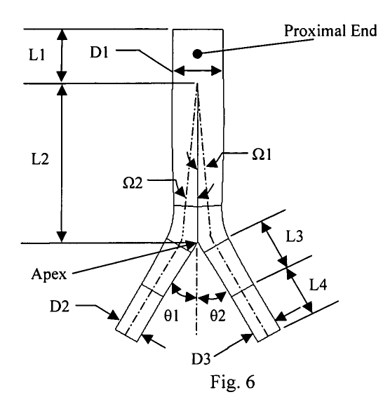

FIG. 6 shows a top view of the various dimensions associated with the blended bifurcated graft. Ω1 and Ω2 vary depending on the distal leg configurations. The three diameters D1, D2 and D3 depend on the vessel diameters at these locations. The angles Ω1 and Ω2 can vary from 0 degrees to 15 degrees and this influences the curve details as given in FIGS. 4 & 5. The angle Ω1 is subtended between the longitudinal axis of the blending section and an axis extending through the centroid of the proximal end of the proximal section and through the centroid of the proximal end of one of the distal legs.

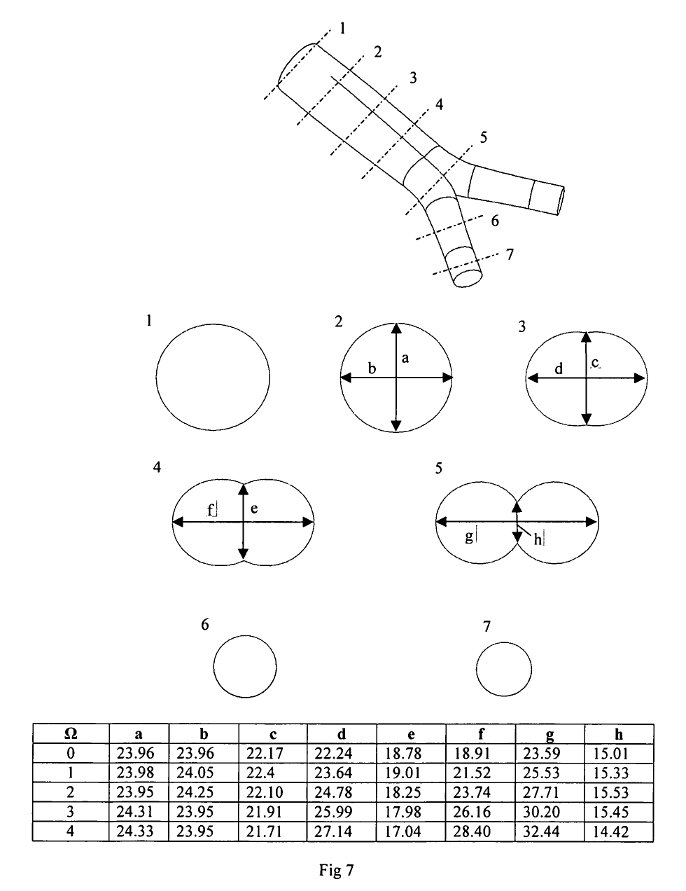

FIG. 7 shows different cross-sections along the blended bifurcation for the proximal section. Section 1 may be circular or elliptical in shape, which can conform to the local geometry of the vessel. The curves for section 1 to 7 can also be circular or elliptical. The sections show the gradual separation of the fluid flow. The table in FIG. 7 gives an example of sizes for average values for the lengths as shown in FIG. 6 with L1=20 mm, L2=90 mm, L3=55 mm with a proximal diameter of 24 mm and iliac leg diameters of 12 mm.

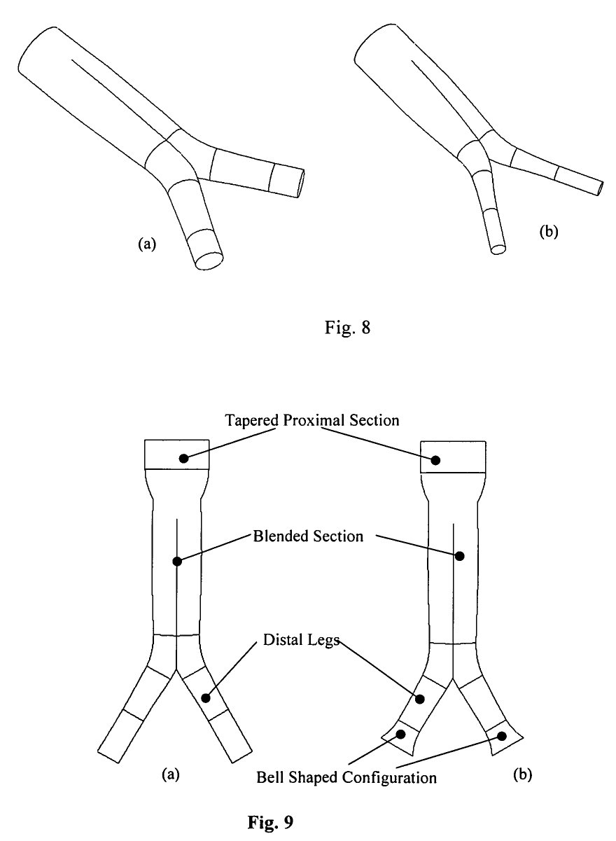

FIGS. 8(a) and 8(b) shows a 3D view of the blended bifurcation for both large and small distal leg configurations. As illustrated in FIG. 8(b), the proximal section and each of the distal legs may taper distally inwardly.

FIGS. 9(a) and 9(b) shows a top view of the blended bifurcation section of a graft with FIG. 9(a) showing a tapered proximal section which can be incorporated and FIG. 9(b) shows bell-shaped distal legs which may need to be added depending on the morphology of the distal vessels. In particular the distal portion of each of the distal legs tapers distally outwardly.

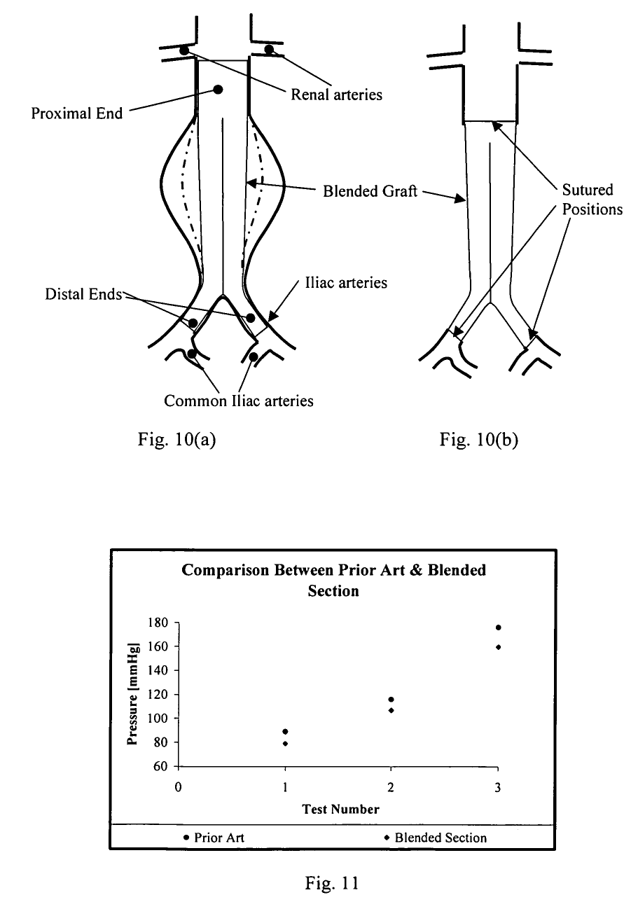

FIG. 10(a) shows the blended section positioned inside an AAA for the endovascular procedure, while FIG. 10(b) shows the blending section being sutured into position between the renal and common iliac arteries for a vascular surgical procedure.

Another variable which minimises the effects of wave reflections is the Young's modulus of the chosen material for the stent and graft. The Young's modulus varies according to the material type and the weaving method chosen i.e. either mono or multi-filament fabric. This implies that there is always a wave reflection due to a change in the elastic properties of a graft or a mismatch in compliance between the host artery and the stent graft. The wave reflection here cannot be totally eliminated, but is minimised by the choice of graft material and stent material that would reduce the difference in mismatch.

The graft is manufactured from biocompatible materials. Monofilament yarn has very high stiffness. The preferred choice of fabric covering is a multi-filament yam or combination of polyester knit/polyurethane material. This fabric reduces the difference in arterial compliance of the diseased artery. The fabric at each attachment site stretches and pulsates with the arterial wall, thus eliminating the need to oversize the fabric. This aids the use of smaller delivery systems. A total pulsating graft in combination with the blending section would minimize the effects of wave reflections being generated.

A preferred stent material is shape memory alloy Nitinol (Nickel/Titanium). This material is one of the most conforming stent materials for attachment against the arterial wall. The lower the Young's modulus of the material the lower the reflection wave will be. A pulsatile fabric or polymer is the preferred option.

For the endovascular procedure the blended graft is positioned below the renal arteries and the right and left common iliac artery as shown in FIG. 4. For the surgical procedure the blended graft is sutured below the renal arteries and above the left and right common iliac arteries as shown in FIG. 5.

The advantages associated with a blending section with the optimized relationship for the area ratios at the bifurcated junction are:
A significant reduction in the reflected forward pressure wave. This avoids the prior art problems of increased proximal blood pressure and reduced flow rate.
Also, by eliminating or reducing reflected pressure waves, continued dilation of the aorta after stent graft placement is reduced or eliminated.
The blending section reduces the drag force by minimizing the effects of the reflected wave.
Due to the increased drag force created by commercial stent grafts high radial force stents with or without hooks and barbs are used. These stents have a significant influence on the dynamic arterial compliance, which creates a material mismatch between the junction of the host artery and stent. This lowering of the compliance generates a condition for the forward wave to be reflected which further increases the proximal blood pressure which leads to a further dilation of the aorta and a further increase in pulsatile drag force. This problem is greatly reduced with a graft of the invention.
This graft of the invention will reduce the need for further anchorage of the proximal end. Prior graft devices either oversize the stent, add hooks and barbs, or use suprarenal stents or a combination of the three. All three approaches have led to problems.
At present in many patients there is a slow and progressive decrease in the aneurysm diameter that leads to a realignment of adjacent vessels and fixation sites. This can cause the proximal neck to vary its angulation and dislodge the stent attachment site, which causes endoleaks. The use of the blending section accommodates the use of a more flexible proximal stent rather than the stiff designs that are available commercially. This flexible proximal stent can adjust easier to any variation in angulation of the proximal neck.
Current endovascular grafts work well in patients with small and medium sized AAAs, however these patients are rarely candidates for surgery. Prior grafts have tried to prevent migration of their devices by making the device as stiff as possible with a fully stented structure. But this columnar strength needed to prevent migration works poorly in tortuous aortas. The graft of the invention aims at reducing the drag forces and reflected pressures instead of stiffening the devices. Stiff devices increase the reflected pressure wave further and increase the chances of migration.
Currently, stent graft devices are only applicable if the proximal diameter is less than 28 mm and the common iliac artery is smaller than 14 mm. Approximately 20% of patients with AAAs have iliac artery aneurysms. Most available stent graft devices do not accommodate iliac aneurysms. This is due to the fact that stent graft devices have standard iliac limb diameters. The surgeon has to combine proximal leg extensions during the operation to achieve the necessary seal in a bell shaped configuration. This bell shaped configuration acts like an expander and is prone to flow separation at the walls, which would eventually lead to blot clotting. The angle of the blending section can be altered to provide an adequate seal past the iliac aneurysm without the use of a bell shaped configuration.
The application of a material with a lower Young's modulus such as a multifilament fabric or a pulsatile fabric or polymer will lower the effects of the reflected wave as well.
The combination of the blending section and pulsatile fabric or polymer create a condition where a more compliant proximal stent can be used. This compliant stent is expected to reduce the effects caused by the material mismatch caused by the host artery and stent.
With a reduction in the reflected wave there will also be a significant drop in the blood pressure. Blood pressure reduction will enhance the medical health of the patient, since there will also be a reduction in the medication requirements.
Stent graft thrombosis, micro-embolism and graft occlusions are two complications associated with endovascular repair of AAA. When area ratio of less than one is employed for the bifurcated junction a sudden contraction of the flow is introduced. This causes the flow to converge which results in a maximum velocity at the junction with minimum pressure. This will subsequently cause flow separation in the iliac legs as the pressure increases due to a decrease in velocity. In order to reduce the foregoing losses, abrupt changes of cross-section should be avoided as is done with the blending section. This blending section prevents flow separation and a reduced vortex circulation. This gives a reduced wall shear stress and consequently reduces the chances of red blood cell damage, which is known to cause graft occlusion.

To test the effects of the graft of the invention over a typical prior device, two rapid prototype parts made from ABS plastic were manufactured. The first part was made to typical commercial shaped geometry while the other incorporated a blending section. A pressure pulse was generated in both models with the same resistance downstream. FIG. 11 shows the results for maximum pressure measured in the proximal end. On average there was a 10% reduction in the proximal pressure with the graft of the invention.

To examine the compliance mismatch effect, two prior stent graft devices were tested in vitro under pulsatile flow conditions and the resulting dynamic displacement was measured by the ME-46 Full Image Video Extensometer (Messphysik GmbH). The Ancure® and Zenith™ stent graft devices were tested experimentally under physiological flow conditions in an idealised and realistic silicone AAA models based on computed tomography scans. There was a considerable reduction in compliance for both stent graft devices which resulted in an increased pulse wave velocity (PWV) and a significant amount of the forward pressure wave being reflected. A reduction in dynamic compliance of 45 and 54% for both the Ancure® and Zenith™ stent graft devices was found respectively. This generated a reflected pressure wave at the proximal stent interface which resulted in 16 and 21% of the forward pulse wave being reflected for the Ancure® and Zenith™ stent graft devices respectively. The blending section reduces the need for high stiffness proximal stents.

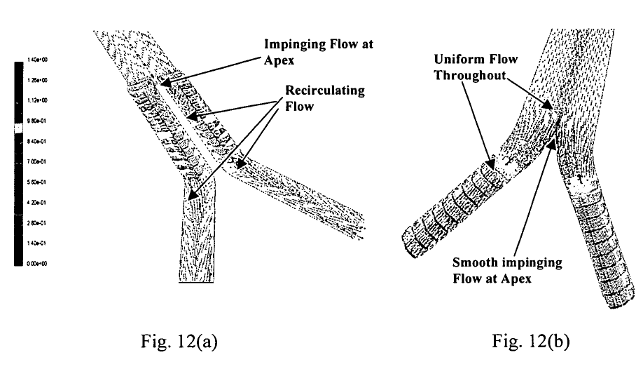

A preliminary Computational Fluid Dynamics (CFD) study was conducted to determine the flow patterns associated with a commercial stent graft and a graft of the invention. FIGS. 12(a) and 12(b) show the axial velocity flow across the centre for both grafts.

As can be seen from FIG. 12(a), the proximal flow first impinges against the bifurcation point which converges the flow downstream of the bifurcation in both iliac legs with a slight recirculation region along the straight portion of the iliac legs. There is a significant recirculating region at the bend in both iliac legs. This bend occurs in prior graft devices when going from the aneurismal sac to both iliac arteries. The blending section as shown in FIG. 12(b) eliminates these recirculation regions by providing a geometry which promotes a greater uniformity of the fluid flow.

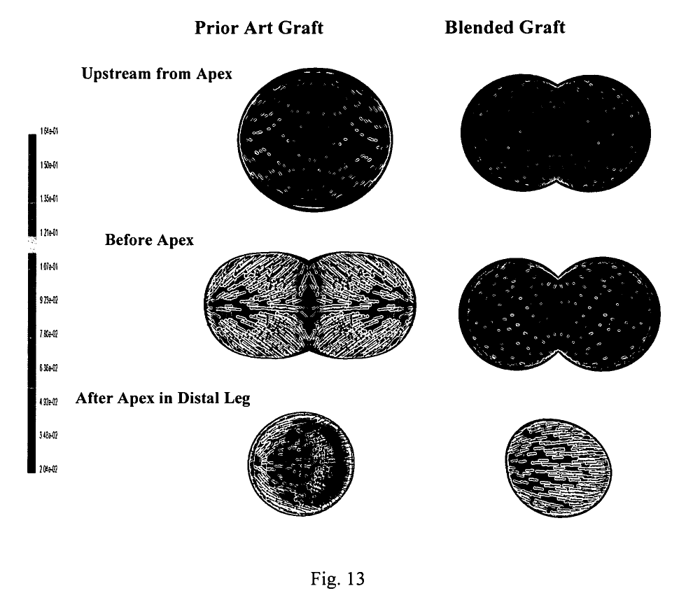

Due to both the blended section and gradual flow separator incorporated in the graft of the invention, there are reduced secondary flows for the blended section graft when compared to the prior art grafts. This is shown by the cross-sectional axial and secondary flow velocities as given in FIG. 13. Upstream from the apex there is little or no secondary flow for both grafts while just before the apex there is a significant increase in secondary flows for the prior art grafts with little or no secondary flows for the blended graft. Due to the incorporation of the gradual flow separator the flow divides in a more parabolic fashion into both distal legs with reduced secondary flows. The prior art grafts create a skewing of the flow with an increased boundary layer before and after the apex in the distal legs. This skewing and increased secondary flows is an undesirable feature which occurs in all prior art devices.

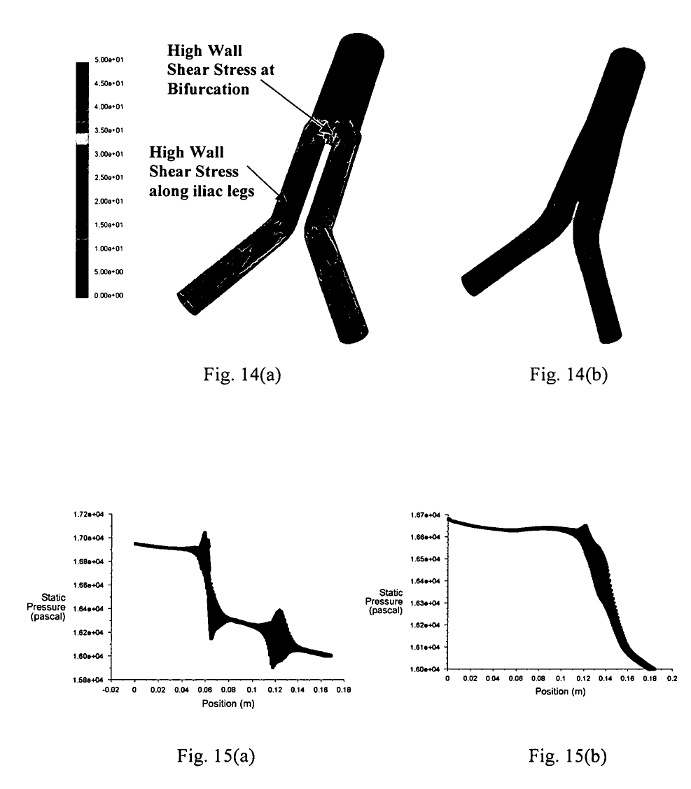

The wall shear stress (WSS) for a prior device as can be seen from FIG. 14(a) is much higher than that for the blending section as shown in FIG. 14(b). This is due to the skewness and recirculation of the flow as was shown in FIGS. 12 & 13, which creates a greater boundary layer for the commercial device when compared to the blended graft.

This high WSS and recirculation region is the main reason for the reported cases of stent graft occlusions and failure of stent graft devices at bifurcation points.

There are two steep decreases in pressure for the commercial stent graft as can be seen from FIG. 15(a). The first occurs at position 0.05 at the bifurcated junction and the second occurs at position 0.12 at the bend in the iliac leg. These steep decreases in pressure are the reason for the recirculation and skewness of the flow as was shown in FIG. 15(a). FIG. 15(b) shows a less severe decrease in pressure along the length of the bifurcation from position 0.12 to 0.16 for the blended stent graft. This explains why there was no recirculation region along the iliac legs and greater uniformity of the flow.

The invention is not limited to the embodiments hereinbefore described, with reference to the accompanying drawings, which may be varied in construction and detail.

1. A vascular graft comprising:
a proximal section;
a first distal leg; and
a second distal leg;
the cross-sectional area of the proximal section being less than or equal to the sum of the cross-sectional area of the first distal leg and the cross-sectional area of the second distal leg.

2. A graft as claimed in claim 1 wherein the graft comprises a blending section between the proximal section and the distal legs.

3. A graft as claimed in claim 2 wherein the blending section defines a first lumen for fluid flow from the proximal section to the first distal leg.

4. A graft as claimed in claim 2 wherein the blending section defines a second lumen for fluid flow from the proximal section to the second distal leg.

5. A graft as claimed in claim 4 wherein the first lumen is separate from the second lumen.

6. A graft as claimed in claim 4 wherein the longitudinal axis of the first lumen is substantially parallel to the longitudinal axis of the second lumen.

7. A graft as claimed in claim 2 wherein the first distal leg and the second distal leg are connected to the blending section at a bifurcation region.

8. A graft as claimed in claim 7 wherein the graft is substantially “Y”-shaped.

9. A graft as claimed in claim 2 wherein at least one of the distal legs is formed integrally with the blending section.

10. A graft as claimed in claim 2 wherein the blending section is formed integrally with the proximal section.

11. A graft as claimed in claim 1 wherein the graft is of integral construction.

12. A graft as claimed in claim 4 wherein the blending section comprises a gradual flow separator to separate flow from the proximal section into the first lumen and into the second lumen.

13. A graft as claimed in claim 2 wherein an apex section is incorporated in the blending section.

14. A graft as claimed in claim 12 wherein the gradual flow separator takes the form of a parabolic, hyperbolic, elliptical, circular, Bezier or B-Spline shape or a combination of these curves.

15. A graft as claimed in claim 13 wherein the apex section takes the form of a parabolic, hyperbolic, elliptical, circular, Bezier or B-Spline shape or a combination of these curves.

16. A graft as claimed in claim 13 wherein the apex section and gradual flow separator are connected as one and take the form of a parabolic, hyperbolic, elliptical, circular, Bezier or B-Spline shape or a combination of these curves.

17. A graft as claimed in claim 2, wherein the blending section provides for a small difference in cross-sectional area between the proximal section and the sum of the cross-sectional areas of the distal legs.

18. A graft as claimed in claim 2 wherein the blending section is between the proximal section and an apex of the graft.

19. A graft as claimed in claim 2 wherein the blending section is incorporated in either one or both of the distal legs.

20. A graft as claimed in claim 12 wherein the cross-section of the proximal section, and/or of the gradual flow separator, and/or of the apex section, and/or of the distal leg is circular, elliptical, parabolic, hyperbolic, Bezier, B-spline shape or a combination of these curves.

21. A graft as claimed in claim 2 wherein the blending section is shaped to minimise pressure wave reflection back to the proximal section.

22. A graft as claimed in claim 2 wherein the blending section is shaped to minimise flow recirculation.

23. A graft as claimed in claim 2 wherein the blending section is shaped to minimise skewing of flow and secondary flow profiles throughout the graft.

24. A graft as claimed in claim 1 wherein at least part of the graft tapers distally inwardly.

25. A graft as claimed in claim 24 wherein at least part of the proximal section tapers distally inwardly.

26. A graft as claimed in claim 24 wherein at least part of the distal leg tapers distally inwardly.

27. A graft as claimed in claim 1 wherein at least part of the graft tapers distally outwardly.

28. A graft as claimed in claim 27 wherein at least part of the distal leg tapers distally outwardly.

29. A graft as claimed in claim 1 wherein the proximal section is tapered.

30. A graft as claimed in claim 1 wherein the distal legs are bell-shaped.

31. A graft as claimed in claim 1 wherein the first distal leg and the second distal leg are substantially symmetrical.

32. A graft as claimed in claim 1 wherein the first distal leg and the second distal leg are substantially asymmetrical.

33. A graft as claimed in claim 1 wherein eccentricity is included.

34. A graft as claimed in claim 2 wherein the angle subtended between:
the longitudinal axis of the blending section; and
an axis extending through the centroid of the proximal end of the proximal section and through the centroid of the proximal end of the distal leg;
is in the range of from 0° to 15°.

35. A graft as claimed in claim 1 wherein the graft is of a material having elasticity properties matching those of a host vessel.

36. A graft as claimed in claim 1 wherein the elasticity properties of the graft varies from 0.1 MPa to 500 MPa.

37. A graft as claimed in claim 1 wherein the elasticity characteristics have viscoelastic or non-linear stress/strain properties.

38. A graft as claimed in claim 1 wherein the graft is of a mono or multi-filament yarn material.

39. A graft as claimed in claim 1 wherein the graft material is a combination of polyester knit and polyurethane or silicone or any other biocompatible rubber or polymer material.

40. A graft as claimed in claim 1 wherein the graft is at least partially of a stretchable material.

41. A graft as claimed in claim 1, wherein the stent material is a shape memory alloy, such as Nitinol, stainless steel or any other biocompatible metal or polymer.

42. A graft as claimed in claim 1, wherein the graft has a stented structure.

43. A graft as claimed in claim 42, wherein the graft has a partially stented structure with stents at the proximal and distal legs.

44. A graft as claimed in claim 1 wherein the graft comprises struts from the proximal section to the distal legs.

45. A graft as claimed in claim 1 wherein the graft comprises a tissue based structure.

46. A graft as claimed in claim 1 wherein the graft is configured for treatment of Abdominal Aortic Aneurysms or any other vascular disease, such as stenois or blocked arteries, or for treatment of blockages in the airways of the trachea entering the lung.

47. A graft as claimed in claim 1, wherein the graft is configured for implantation by vascular surgery.

48. A graft as claimed in claim 1, wherein the graft is configured for implantation by endovascular surgery.

49. A graft as claimed in claim 1, wherein the graft is modular, having different sized sections for the proximal and both distal legs exist for general and patient-specific anatomy sizes.

50. A vascular graft comprising:
a proximal section;
a first distal leg;
a second distal leg; and
a blending section between the proximal section and the distal legs;
the first distal leg and the second distal leg being connected to the blending section at a bifurcation region;
the blending section defining a first lumen for fluid flow from the proximal section to the first distal leg and a second lumen for fluid flow from the proximal section to the second distal leg, the first lumen being separate from the second lumen;
at least one of the distal legs being formed integrally with the blending section.
